(19) United States
(12) Patent Application Publication
(10) Pub. No.: US 2010/0323379 A1
(43) Pub. Date: Dec. 23, 2010

(54) SLEEP APNEA

(76) Inventors: Virend K. Somers, Rochester, MN (US); Michal S. Hoffmann, Rochester, MN (US); Robert Wolk, Salem, CT (US)

Correspondence Address: FISH & RICHARDSON P.C. (TC), PO BOX 1022, MINNEAPOLIS, MN, 55440-1022, US

(21) Appl. No.: 12/680,073

(22) PCT Filed: Sep. 25, 2008
(86) PCT No.: PCT/US08/77608

§ 371 Date: Sep. 8, 2010

Related U.S. Application Data
(60) Provisional application No. 60/975,699, filed on Sep. 27, 2007.

(51) Int. Cl. G01N 33/50; G01N 33/68; C12Q 1/34; C12Q 1/30; C12Q 1/26; C12Q 1/48; C12Q 1/32; C12Q 1/28

(52) U.S. Cl. 435/15; 436/86; 436/94; 435/18; 435/27; 435/25; 435/26; 435/28

(57) ABSTRACT

This document relates to methods and materials involved in diagnosing sleep apnea and assessing the effectiveness of a treatment for sleep apnea. For example, methods and materials for using markers to determine whether or not a mammal (e.g., a human) has sleep apnea are provided. In addition, methods and materials that can be used to determine whether or not a mammal (e.g., a human) responds to a sleep apnea treatment are provided.

### CROSS-REFERENCE TO RELATED APPLICATIONS

This application claims benefit of priority from U.S. Provisional Application Ser. No. 60/975,699, filed on Sep. 27, 2007.

### STATEMENT AS TO FEDERALLY SPONSORED RESEARCH

This invention was made with government support under grant number HL065176 awarded by National Institutes of Health. The government has certain rights in the invention.

### BACKGROUND

1. Technical Field

This document relates to methods and materials involved in diagnosing sleep apnea and assessing the effectiveness of a treatment for sleep apnea.

2. Background Information

Obstructive sleep apnea (OSA) is a common sleep-related breathing disorder, with an approximately 25% prevalence in the adult US population (Young et al., N. Engl. J. Med., 328:1230-1235 (1993)). Central sleep apnea (CSA) is also linked to cardiovascular disease and early mortality in heart failure patients. OSA and CSA are characterized by recurrent episodes of cessation of respiratory airflow during sleep leading to sleep fragmentation and periodic and often severe intermittent hypoxia (IH). OSA has been linked to an increased incidence of cardiovascular diseases, including hypertension, atrial fibrillation, arrhythmias, coronary artery disease, and heart failure. Sympathetic activation and systemic inflammation elicited by OSA have been implicated as possible causes of increased cardiovascular risk.

### SUMMARY

This document relates to methods and materials involved in diagnosing sleep apnea (e.g., obstructive or central sleep apnea) and assessing the effectiveness of a treatment for sleep apnea. For example, this document provides methods and materials for using biomarkers to determine whether or not a mammal (e.g., a human) has sleep apnea. In addition, this document provides methods and materials that can be used to determine whether or not a mammal (e.g., a human) responds to a sleep apnea treatment. For example, a human receiving a sleep apnea treatment (e.g., a continuous positive airway pressure (CPAP) and/or postural adjustments) who is found have serum that does not contain evidence of the differential expression of a nucleic acid and/or polypeptide such as, but not limited to, those listed in Table 1 during or after sleep as compared to the level before sleep can be classified as responding to that sleep apnea treatment. This document also provides arrays for detecting polypeptide or nucleic acid levels that can be used to diagnose sleep apnea in a mammal. Such arrays can allow clinicians to diagnose sleep apnea based on a determination of the levels of nucleic acids and polypeptides that are differentially regulated in sleep apnea patients as compared to healthy controls.

This document is based, in part, on the discovery of molecules (e.g., nucleic acids) that are differentially regulated between sleep apnea patients and healthy controls. This document also is based, in part, on the discovery that the levels of nucleic acid expression before and after sleep can be used to distinguish mammals with sleep apnea from healthy mammals. For example, the levels of mRNA for the nucleic acids listed in Table 1 can be assessed to diagnose sleep apnea. In some cases, a mammal (e.g., a human) can be assessed to determine whether or not the mammal contains a sleep apnea signature. The presence of a sleep apnea signature can indicate that the mammal suffers from sleep apnea. For the purpose of this document, the term “sleep apnea signature” as used herein refers to a nucleic acid or polypeptide expression profile where one or more (e.g., two, three, four, five, six, seven, eight nine, ten, 15, or more) nucleic acids or polypeptides such as, but not limited to, those listed in Table 1 are present at a level greater than or less than the level observed in a control sample from a control mammal. In some cases, the sleep apnea signature can be a nucleic acid or polypeptide profile where 10, 20, 30, 40, 50, 60, 70, 80, 90, or 100 percent of the nucleic acids or polypeptides listed in Table 1 are present at a level greater than or less than the level observed in a control sample from a control mammal, when measured before and after sleep.

In general, this document features a method for identifying a mammal having sleep apnea. The method comprises determining whether or not a mammal comprises a sleep apnea signature, wherein the presence of the sleep apnea signature indicates that the mammal has sleep apnea. The mammal can be a human. The method can comprise determining whether or not a blood sample from the mammal comprises the sleep apnea signature. The method can comprise determining whether or not a urine, saliva, or perspiration sample from the mammal comprises the sleep apnea signature. The method can comprise determining whether or not breath from the mammal comprises the sleep apnea signature. The method can comprise determining whether or not the mammal comprises the sleep apnea signature based on expression level changes before and after sleep.

In another aspect, this document features a method for identifying a mammal having sleep apnea. The method comprises determining whether or not a mammal comprises a level of expression of a nucleic acid or a polypeptide indicative of sleep apnea, wherein the presence of the level indicates that the mammal has sleep apnea. The nucleic acid or the polypeptide can be listed in Table 1. The mammal can be a human. The method can comprise determining whether or not a blood sample from the mammal comprises the level. The method can comprises determining whether or not a urine, saliva, or perspiration sample from the mammal comprises the level of expression.

In another aspect, this document features a method for assessing the effectiveness of a treatment for sleep apnea. The method comprises determining whether or not the level of expression of a nucleic acid or polypeptide in a mammal being treated for sleep apnea changes during sleep, wherein a change in the level during sleep indicates that the treatment is ineffective. The nucleic acid or the polypeptide can be listed in Table 1. The mammal can be a human. The method can comprise assessing a blood sample obtained from the mammal. The method can comprise assessing a urine, saliva, or perspiration sample obtained from the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Expression profiles of nucleic acids involved in ROS modulation in healthy controls (grey bars) and OSA subjects (black bars). Left panels show measurements at 9 pm and 6 am. Right panels show overnight % changes. ‡p<0.05 for OSA vs. controls at baseline (9 pm); # p<0.05 for overnight % changes in OSA vs. controls; *p>0.05<0.10 for overnight % changes in OSA vs. controls.

FIG. 2. Expression profiles of nucleic acids involved in ROS modulation in healthy controls (grey bars) and OSA subjects (black bars).

FIG. 3. Expression profiles of nucleic acids involved in cell growth, proliferation, or cell cycle in healthy controls (grey bars) and OSA subjects (black bars). Left panels show measurements at 9 pm and 6 am. Right panels show overnight % changes. ‡p<0.05 for OSA vs. controls at baseline (9 pm); # p<0.05 for overnight % changes in OSA vs. controls.

FIG. 4. Expression profiles of nucleic acids involved in cell growth, proliferation, or cell cycle in healthy controls (grey bars) and OSA subjects (black bars).

FIG. 5. DUSP-1 nucleic acid expression in rt-qPCR normalized and presented as a percentage of DUSP to beta actin expression ratio.

FIG. 6. The influence of CPAP treatment on DUSP-1 nucleic acid expression in rt qPCR analysis.

FIG. 7 is a graph plotting the level of DUSP1 nucleic acid expression at the indicated times for control and OSA subjects.

FIG. 8 is a graph plotting the level of RAF1 nucleic acid expression at the indicated times for control and OSA subjects.

FIG. 9 is a graph plotting the level of MAP2K2 nucleic acid expression at the indicated times for control and OSA subjects.

FIG. 10 is a graph plotting the level of SLAP nucleic acid expression at the indicated times for control and OSA subjects.

FIG. 11 is a graph plotting the level of eIF4EBP nucleic acid expression at the indicated times for control and OSA subjects.

FIG. 12 is a graph plotting the level of TEF2 nucleic acid expression at the indicated times for control and OSA subjects.

FIG. 13 is a graph plotting the level of Sel-1 nucleic acid expression at the indicated times for control and OSA subjects.

FIG. 14 is a graph plotting the level of PI4 Kb nucleic acid expression at the indicated times for control and OSA subjects.

FIG. 15 is a graph plotting the level of 5 Inositol polyphosphate phosphatase nucleic acid expression at the indicated times for control and OSA subjects.

FIG. 16 is a graph plotting the level of CD86 nucleic acid expression at the indicated times for control and OSA subjects.

FIG. 17 is a graph plotting the level of cdc25b nucleic acid expression at the indicated times for control and OSA subjects.

FIG. 18 is a graph plotting the level of IKK alpha nucleic acid expression at the indicated times for control and OSA subjects.

FIG. 19 is a graph plotting the level of NFkappaB inhibitor nucleic acid expression at the indicated times for control and OSA subjects.

FIG. 20 is a graph plotting the level of casein kinase 1 gamma 2 nucleic acid expression at the indicated times for control and OSA subjects.

FIG. 21 is a graph plotting the level of PGS1 nucleic acid expression at the indicated times for control and OSA subjects.

FIG. 22 is a graph plotting the level of LDH B nucleic acid expression at the indicated times for control and OSA subjects.

FIG. 23 is a graph plotting the level of CXCR4 nucleic acid expression at the indicated times for control and OSA subjects.

FIG. 24 is a graph plotting the level of IL-13 receptor nucleic acid expression at the indicated times for control and OSA subjects.

FIG. 25 is a graph plotting the level of RALB nucleic acid expression at the indicated times for control and OSA subjects.

FIG. 26 contains graphs plotting the level of nucleic acid expression for the indicated nucleic acids at the indicated times for healthy control subjects (grey bars) and OSA subjects (black bars).

### DETAILED DESCRIPTION

This document relates to methods and materials involved in diagnosing sleep apnea and assessing the effectiveness of a treatment for sleep apnea. As described herein, this document provides methods and materials for diagnosing a mammal (e.g., a human) as having sleep apnea. In some embodiments, a mammal can be diagnosed as having sleep apnea if it is determined that a sample from the mammal (e.g., a blood sample) contains one or more of the nucleic acids or polypeptides listed in Table 1 at a level that is greater than or less than the average level of the same one or more nucleic acids or polypeptides observed in a control sample obtained from a control mammal. In some cases, measurements can be obtained before and immediately after sleep, and if necessary, several hours after waking. In general, when compared to normal sleep, sleep apnea induces differences in magnitude and/or direction of change of one or more markers, when they are measured before and immediately after sleep. In some cases, changes can resolve when re-measured several hours after waking. Similarly, effective therapy of sleep apnea (e.g., OSA) can be evidenced by attenuation of the magnitude or directionality of the change in biomarkers elicited by sleep apnea. As described herein, biomarker levels can change after a period of normal sleep, sleep apnea, and treated sleep apnea, allowing assessment of the presence or absence, and/or effective treatment of, sleep apnea.

TABLE 1
Nucleic acids differentially expressed in mammals having sleep apnea.

| | GenBank GI | Over-expressed or under-expressed as |
|---|---|---|
| Nucleic acid | Accession No. | compared to normal controls |
| Dual specificity | GI: 7108342 | Over-expressed after overnight |
| phosphatase 1 | NM_004417 | sleep apnea versus controls |
| DUSP1 | | |
| nuclear factor of kappa | GI: 10092618 | Over-expressed after overnight |
| light polypeptide gene | NM_020529 | sleep apnea versus controls |
| enhancer in B-cells | | |
| inhibitor, alpha | | |
| NFKBIA | | |
| catalase | GI: 60302919 | Over-expressed after overnight |
| | NM_001752 | sleep apnea versus controls |
| chemokine (C—X—C motif) | GI: 56790928 | Over-expressed after overnight |
| receptor 4 | NM_003467 | sleep apnea versus controls |
| CXCR4 | | |
| v-ral simian leukemia | GI: 48762927 | Over-expressed after overnight |
| viral oncogene homolog B | NM_002881 | sleep apnea versus controls |
| (ras related; GTP binding | | |
| protein) RALB | | |
| eukaryotic translation | GI: 31542585 | Over-expressed after overnight |
| initiation factor 4E | NM_004096 | sleep apnea versus controls |
| binding protein 2 | | |
| (EIF4EBP2) | | |
| Phosphatidylglycerophosphate | GI: 84508630 | Over-expressed after overnight |
| Synthase (PGS1) | NM_024419 | sleep apnea versus controls |
| CCAATenhancer binding | GI: 28872795 | Over-expressed in sleep apnea |
| protein (CEBP), beta | NM_005194 | versus controls |
| CEBPB | | |
| interleukin 13 receptor, | GI: 26787975 | Over-expressed after overnight |
| alpha 1 (IL13RA1) | NM_001560 | sleep apnea versus controls |
| casein kinase 1, gamma 2 | GI: 2199528 | Over-expressed after overnight |
| CSNK1G2 | AF001177 | sleep apnea versus controls |
| Human Src-like adapter | GI: 113930753 | Over-expressed after overnight |
| protein | NM_001045556 | sleep apnea versus controls |
| SLA | | |
| cargo selection protein | GI: 4206367 | Over-expressed in sleep apnea |
| (mannose 6 phosphate | AF051314 | versus controls |
| receptor binding protein) | | |
| (TIP47) | | |
| RNA-binding protein | GI: 1568642 | Over-expressed in sleep apnea |
| BRUNOL3 (BRUNOL3) | U69546 | versus control |
| CUGBP2 | | |
| sel-1 (suppressor of lin-12, | GI: 19923668 | Over-expressed after overnight |
| C. elegans)-like | NM_005065 | sleep apnea versus controls |
| v-raf-1 murine leukemia | GI: 52486392 | Over-expressed (sleep apnea after |
| viral oncogene homolog 1 | NM_002880 | sleep vs sleep apnea before sleep) |
| (RAF1) | | |
| B-cell translocation gene | GI: 4502472 | Over-expressed (sleep apnea after |
| 1, anti-proliferative | NM_001731 | sleep vs sleep apnea before sleep) |
| (BTG1) | | |
| inositol polyphosphate-5- | NM_005539 | Over-expressed (sleep apnea after |
| phosphatase, 40 kD | XM_936595 | sleep vs sleep apnea before sleep) |
| (INPP5A) | GI: 109702905 | |
| Heme oxygenase 1 | GI: 4504436 | Over-expressed (sleep apnea vs |
| HMOX1 | NM_002133 | healthy control before sleep) |
| | | Under-expressed (sleep apnea |
| | | after sleep vs sleep apnea before |
| | | sleep |
| Superoxide dysmutase 1 | GI: 48762945 | Over-expressed (sleep apnea vs |
| SOD1 | NM_000454 | healthy control before sleep) |
| | | Under-expressed (sleep apnea |
| | | after sleep vs sleep apnea before |
| | | sleep |
| carcinoembryonic antigen- | GI: 219538 | Under-expressed |
| related cell adhesion | D90276 | |
| molecule 4 (CEACAM4) | | |
| Lactate dehydrogenase 1 | GI: 89161189 | Over-expressed (sleep apnea vs |
| LDHB | AC_000055 | control before sleep) Under- |
| | | expressed (sleep apnea after sleep |
| | | vs sleep apnea before sleep |
| Homo sapiens mitogen- | GI: 307184 | Over-expressed (sleep apnea vs |
| activated protein kinase | L11285 | control before sleep) Under- |
| kinase 2 (MAP2K2) | | expressed (sleep apnea after sleep |
| | | vs sleep apnea before sleep) |
| CD86 | GI: 91208432 | Under-expressed |
| | NM_006889 | |
| CDC25B cell division | GI: 47078253 | Over-expressed (sleep apnea vs |
| cycle 25 homolog B | NM_021874 | healthy control before sleep) |
| CDC25B | | Under-expressed (sleep apnea |
| | | after sleep vs sleep apnea before |
| | | sleep |

The mammal can be any mammal such as a human, dog, mouse, or rat. Any method can be used to obtain a sample (e.g., blood, saliva, urine, perspiration, and/or expired air) for evaluation. For example, a sample such as blood can be obtained by peripheral venipuncture and evaluated to determine if it contains (1) one or more of nucleic acids or polypeptides, such as those listed in Table 1, at a level that is greater than or less than the average level observed in a control sample. The level of any number of nucleic acids or polypeptides such as those listed in Table 1 can be evaluated to diagnose sleep apnea. For example, the level of one or more than one (e.g., two, three, four, five, six, seven, eight, nine, ten, 15, or more than 30) of the nucleic acids or polypeptides listed in Table 1 can be measured before and after sleep.

The level of expression can be greater than or less than the average level observed in a control sample obtained from one or more control mammals. Typically, a nucleic acid or polypeptide can be classified as being present at a level that is greater than or less than the average level observed in a control sample if the levels differ by at least 10, 20, 30, 40, 50, 60, 70, 80, 90, or more percent. In some cases, a nucleic acid or polypeptide can be classified as being present at a level that is greater than or less than the average level observed in a control sample if the levels differ by greater than 1-fold (e.g., 1.5-fold, 2-fold, 3-fold, or more than 3-fold). Control samples typically are of the same species as the mammal being evaluated. In some cases, a control sample can be obtained from one or more mammals that are from the same species as the mammal being evaluated. When diagnosing sleep apnea, control blood samples can be isolated from healthy mammals such as healthy humans who do not have sleep apnea. Any number of control mammals can be used to obtain the control serum. For example, control blood samples can be obtained from one or more healthy mammals (e.g., at least 5, at least 10, at least 15, at least 20, or more than 20 control mammals). The control measurements can be made both before and after sleep.

Any method can be used to determine whether or not a nucleic acid or polypeptide is present at a level that is greater than or less than the average level observed in a control sample. For example, the level of a particular nucleic acid (e.g., mRNA) can be measured using, without limitation, PCR-based assays such as real-time PCR. Methods of using arrays for detecting nucleic acids include, without limitation, those described herein. Such methods can be used to determine simultaneously the relative levels of multiple nucleic acids. The level of a particular polypeptide can be measured using, without limitation, immuno-based assays (e.g., ELISA), western blotting, arrays for detecting polypeptides, two-dimensional gel analysis, chromatographic separation, or mass spectroscopy. Methods of using arrays for detecting polypeptides include, without limitation, those described herein. Such methods can be used to determine simultaneously the relative levels of multiple polypeptides.

A mammal can also be assessed using the methods and materials provided herein before, during, and after being treated for sleep apnea. Assessing a mammal during treatment of the mammal for sleep apnea can allow the effectiveness of the sleep apnea therapy to be determined.

This document also provides arrays for detecting nucleic acids or polypeptides. The arrays provided herein can be two-dimensional arrays, and can contain at least two different nucleic acids (e.g., nucleic acid probes) or polypeptides (e.g., antibodies) capable of detecting nucleic acids or polypeptides (e.g., at least three, at least five, at least ten, at least 20, at least 30, at least 50, at least 100, or at least 200 different nucleic acids or polypeptides capable of detecting nucleic acids or polypeptides). The arrays provided herein also can contain multiple copies of each of many different nucleic acids or polypeptides. In addition, the arrays for detecting nucleic acids or polypeptides provided herein can contain nucleic acids or polypeptides attached to any suitable surface (e.g., plastic or glass).

A polypeptide capable of detecting a polypeptide can be naturally occurring, recombinant, or synthetic. The polypeptides immobilized on an array also can be antibodies or antibody fragments, such as Fab′ fragments, Fab fragments, single-chain Fvs, antigen-specific polyclonal antibodies, or full-length monoclonal antibodies. Such an antibody or antibody fragment can be capable of binding specifically to a polypeptide listed in Table 2, 3, or 4. The polypeptides immobilized on the array can be members of a family such as a receptor family, ligand family, or enzyme family.

The production of monoclonal antibodies against a polypeptide target is routine using standard hybridoma technology. In addition, numerous monoclonal antibodies are available commercially. An antibody fragment can be produced by any means. For example, an antibody fragment can be enzymatically or chemically produced by fragmentation of an intact antibody. An antibody fragment also can be produced synthetically or recombinantly from a gene encoding the partial antibody sequence. The antibody fragment can be a single chain antibody fragment. Alternatively, the fragment can include multiple chains which are linked together, for instance, by disulfide linkages. The fragment may also optionally be a multimolecular complex.

Any method can be used to make an array for detecting polypeptides. For example, methods disclosed in U.S. Pat. No. 6,630,358 can be used to make arrays for detecting polypeptides. Arrays for detecting polypeptides can also be obtained commercially, such as from Panomics, Redwood City, Calif.

The measurement of biomarkers obtained from blood, saliva, urine, perspiration and/or expired air can be used as an index of the presence of sleep apnea. Measurements obtained before and after untreated sleep apnea and changes in the biomarkers can be indicative of the presence and severity of sleep apnea. Measurements of biomarkers before and after treatment of sleep apnea overnight can provide an index of whether or not sleep apnea treatment is effective. Absence of change in biomarkers can indicate effective treatment, and the magnitude of any change can be indicative of inadequacy of treatment. These measurements, which can be obtained before and after sleep with untreated sleep apnea, or before and after sleep with treated sleep apnea, can be obtained on repeated occasions given their safety and ease of acquisition. Therefore, in any individual, several measurements can be obtained on different days, before and after sleep, or before and after a period of untreated sleep apnea, to determine whether or not sleep apnea is present. Over the course of treatment, monitoring of treatment effectiveness can be also ascertained by measuring the levels of the markers provided herein.

In some cases, measurements can be obtained immediately after waking from sleep and repeated at a variable time later. A change in the markers over the time of wakefulness can indicate the presence of sleep apnea or the presence of incompletely treated sleep apnea.

The invention will be further described in the following examples, which do not limit the scope of the invention described in the claims.

### EXAMPLES

### Biomarkers for Sleep Apnea

### Human Subjects

Eight male subjects were recruited: four healthy controls and four patients with newly diagnosed, never-treated, severe OSA (AHI>30), without any other co-morbidities. None of the subjects was smoking or taking any medications. Patients and controls were of similar age and had similar body mass index (BMI; Table 2). Subjects were admitted at 5.30 pm, and underwent full polysomnography. Blood was collected at two time points, before sleep (at 9 pm, 4 hours after the last meal), and then directly after waking from sleep (at 6 am).

TABLE 2
Baseline characteristics of healthy controls vs. OSA patients.

| | Healthy (N = 4) | OSA (N = 4) | P |
|---|---|---|---|
| Age | 37.5 ± 5.06 | 40.7 ± 9.59 | NS |
| BMI (kg/m²) | 32.6 ± 3.59 | 35.5 ± 1.29 | NS |
| Basal O₂ sat | 96.9 ± 0.8 | 96.75 ± 0.5 | NS |
| AHI, events/hr | 0.6 ± 0.7 | 50.25 ± 23.5 | 0.0001 |
| Mean nocturnal O₂ sat | 96.2 ± 1.02 | 93.75 ± 2.12 | 0.001 |
| Lowest nocturnal O₂ sat | 92.16 ± 3.68 | 59.5 ± 17.13 | 0.005 |
| % of sleep time with Hb | 100 | 85.75 ± 10.24 | 0.03 |
| sat >90% | | | |

NS indicates not significant

### Microarray Experiment

The blood containing tubes were initially incubated at room temperature for three hours to stabilize cellular RNA followed by its isolation using PAX Gene RNA isolation kit (Qiagen, Chatsworth, Calif.) according to manufacturer's instructions, and then processed for microarray experiments as described elsewhere (Sreekumar et al., Diabetes, 51:1913-1920 (2002)). Briefly, total RNA (2 μg) was converted to cDNA using the Superscript cDNA synthesis kit (Gibco-BRL, Gaithersberg, Md.). Double stranded cDNA was then purified by phase lock gel (Eppendorf, Westbury, N.Y.) with phenol/chloroform extraction. The purified cDNA was used as a template for in vitro transcription reaction for the synthesis of biotinylated cRNA using RNA transcript labeling reagent (Affymetrix, Santa Clara, Calif.). These labeled cRNAs were then fragmented and hybridized onto the HG-U133A and B arrays (Affymetrix, Santa Clara, Calif.). Following hybridization, the solutions were removed, and the arrays were washed and stained with streptavidin-phycoerythrin (Molecular Probes, OR).

Following washes, arrays were scanned using GeneChip scanner 3000 made by Hewlett-Packard.

### Microarray Data Analysis

The microarray data were analyzed using Spotfire™ 7.2, commercially available software. The level of gene expression for each subgroup was presented as an average with standard deviation.

### Statistical Analysis

The treatment comparison application using ANOVA was used in order to identify statistically significant differences in gene expression among the groups. Data are presented as mean±SD for continuous variables and as number and percentages for categorical variables. Paired and unpaired two-sample equal variance Student's t-test were used to determine statistical significance of differences and changes between and within the study groups, respectively. P values<0.05 were considered statistically significant.

### Results

The baseline characteristics and sleep profiles of the control subjects and OSA patients are shown in Table 2. Only sleep profiles (specifically nocturnal oxygen saturation and AHI) differed significantly between the two groups, indicating the presence of severe oxygen desaturations and apneic events in the OSA group.

Using ANOVA analysis of the microarray data before and after sleep, significant differences in gene expression between subjects with and without OSA were identified, as were several sleep-induced changes in gene expression. The observed differences and changes were related to genes encoding anti-oxidant enzymes (Table 3), as well as polypeptides involved in cell cycle regulation and growth (Table 4).

TABLE 3
Genes involved in modulation of reactive oxygen species, and function of
the encoded polypeptides.

| | | Baseline | Overnight |
|---|---|---|---|
| | | OSA vs. | % change |
| | | Control (at | OSA vs. |
| Name of gene | Function of encoded polypeptide | 9 pm) | Control |
| Heme oxygenase1 | a 32-kDa heat shock protein, induced by stress and | ↑, 0.01 | ↓, 0.02 |
| (HMOX1) | protects cell from oxidative damage (Platt and | | |
| | Nath, Nat. Med., 4: 1364-1365 (1998)) | | |
| Superoxide | endogenous antioxidant in mammalian cells, | ↑, 0.008 | ↓, 0.02 |
| dismutase 1 (SOD1, | catalyzes the dismutation of the superoxide anion | | |
| Cu/Zn) | (O₂⁻) into hydrogen peroxide and molecular | | |
| | oxygen (Dimayuga et al., J. Neuroimmunol., | | |
| | 182: 89-99 (2007)) | | |
| Superoxide | involved in defense against toxic superoxide (O₂⁻), | ↓, NS, 0.38 | ↑, 0.008 |
| dismutase 2 (SOD2, | protects macrophages from oxidative stress | | |
| Mn) | (Rakkola et al., Proteomics, 7(3): 378-84 (2007) | | |
| Catalase | antioxidant enzyme that detoxifies hydrogen | ↑, NS, 0.53 | ↑, 0.10 |
| | peroxide by converting it to water and oxygen, | | |
| | thereby preventing cellular injury (Rohrdanz and | | |
| | Kahl, Free Radic. Biol. Med., 24: 27-38 (1998)) | | |
| Peroxiredoxin 5 | plays a role in protecting the genome against | NS, 0.11 | NS, 0.97 |
| | oxidation (Kropotov et al., FEBS J., 273: 2607-2617 | | |
| | (2006)) | | |
| Peroxiredoxin 4 | induces in a stress-specific fashion to protect | NS, 0.37 | NS, 0.39 |
| | human cells from oxidant injury (Shen and | | |
| | Nathan, Mol. Med., 8: 95-102 (2002)) | | |
| Thioredoxin | member of a family of pyridine nucleotide | NS, 0.67 | NS, 0.16 |
| reductase | oxidoreductases, plays a role in protection against | | |
| | oxidative stress (Hashemy, J. Biol. Chem., | | |
| | 281: 10691-10697 (2006)) | | |
| Thioredoxin | multiple functions in regulation of cell growth, | NS, 0.45 | NS, 0.18 |
| | apoptosis, and activation, constitutes an | | |
| | endogenous antioxidant system (Powis and | | |
| | Montfort, Annu. Rev. Biophys. Biomol. Struct., | | |
| | 30: 421-455 (2001)) | | |
| Thioredoxin | inhibits antioxidative function by inhibition of the | NS, 0.83 | NS, 0.23 |
| interacting protein | thioredoxin ROS-scavenging system (Junn et al., J. | | |
| (TXNIP) | Immunol., 164: 6287-6295 (2000)) | | |
| Glutathione | member of the glutathione peroxidase family, | NS, 0.98 | NS, 0.45 |
| peroxidase 1 | functions in the detoxification of hydrogen | | |
| | peroxide, and is an important antioxidant enzyme | | |
| | in humans (Arthur, Cell Mol. Life Sci., 57: 1825-1835 | | |
| | (2000)) | | |

NS indicates not significant

TABLE 4
Genes modulating the cell cycle and function of encoded polypeptides.

| | | Baseline OSA | Overnight % |
|---|---|---|---|
| | | vs. Control | change OSA vs. |
| Name of gene | Function of encoded polypeptide | (at 9 pm) | Control |
| Ribonucleotide | involved in DNA synthesis crucial for | NS, 0.17 | NS 0.44 |
| reductase M1 | S phase of cell cycle (Jordan and | | |
| polypeptide | Reichard, Annu. Rev. Biochem., 67: 71-98 | | |
| | (1998)) | | |
| Cell division cycle 25 | belongs to CDC25 phosphatases | ↑, 0.01 | ↓, 0.01 |
| B (CDC25B) | family, activates CDC2 thus enabling | | |
| | the cell to enter the mitotic phase | | |
| | (Turowski et al., Mol. Biol. Cell, | | |
| | 14: 2984-2998 (2003)) | | |
| Eukaryotic translation | protein translation Gebauer and Hentze, | NS, 0.59 | NS, 0.14 |
| elongation factor2 | Nat. Rev. Mol. Cell Biol., 5: 827-835 | | |
| | (2004) | | |
| Signaling lymphocyte | co-stimulates T lymphocyte | ↑, 0.10 | ↓, 0.03 |
| activating molecule | proliferation and IFN gamma synthesis | | |
| (SLAM) | (Watts and DeBenedette, Curr. Opin. | | |
| | Immunol., 11: 286-293 (1999)) | | |
| Calgizzarin S100A11 | calcium induced growth inhibitor | NS, 0.68 | ↑, 0.04 |
| | (Sakaguchi et al., J. Cell. | | |
| | Biol., 163: 825-835 (2003)) | | |
| B-cell translocation | inhibits cell cycle in G0/G1 phase | NS, 0.61 | NS, 0.43 |
| gene | (Rouault et al., Embo J., 11: 1663-1670 | | |
| | (1992)) | | |
| Src-like adapter | participates in T cell receptor signal | NS, 0.88 | ↑, 0.05 |
| protein (SLAP) | transduction, negative mitosis regulator | | |
| | (Roche et al., Curr. Biol., 8: 975-978 | | |
| | (1998) and Sosinowski et al., J. Exp. | | |
| | Med., 191: 463-474 (2000)) | | |
| Eukaryotic translation | binds to eIF4E and inhibits protein | ↓, NS, 0.13 | ↑, 0.00007 |
| initiation factor 4E | translation (Gebauer and Hentze, Nat. | | |
| binding protein | Rev. Mol. Cell Biol., 5: 827-835 (2004)) | | |
| Supressor of Lin-12 | inhibits transcription of factors | NS, 0.97 | NS, 0.18 |
| C. elegans-like (Sel1) | responsible for the cell growth | | |
| | (Cattaneo et al., Gene, 326: 149-156 | | |
| | (2004)) | | |

NS indicates not significant

### Genes Related to Oxidative Stress

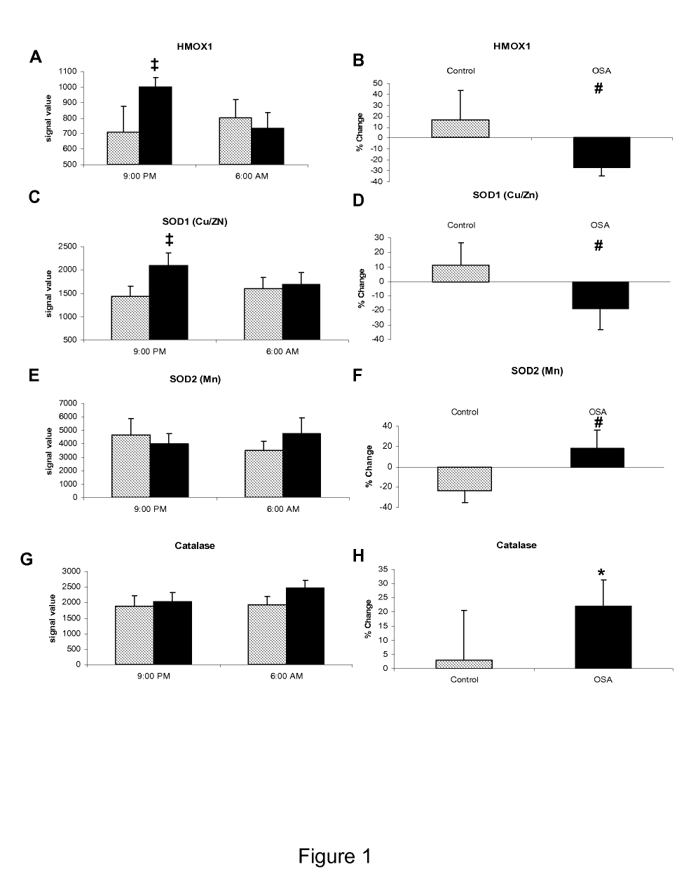

The gene transcript levels of heme oxygenase 1(HMOX1) gene activity measured at night (9 pm) were higher in OSA patients than in healthy subjects (p=0.01). However, HMOXI gene activity in OSA patients decreased during sleep to the same levels as observed in healthy controls (change in OSA vs. change in controls: p=0.021) (FIGS. 1A and 1B).

The gene transcript levels of superoxide dismutase 1 (cytoplasmic isoform: Cu/Zn, SOD1) at night were also much higher in OSA patients (OSA vs. controls, p=0.008) than in healthy controls. SOD1 transcript levels also declined overnight in OSA patients, but remained unchanged in controls (p=0.02) (FIGS. 1C and 1D). Superoxide dismutase 2 (mitochondrial isoform: Mn, SOD2) had similar baseline activity in controls and OSA subjects. In the control group, the gene transcript level decreased during sleep (p=0.06), whereas SOD2 activity increased after repetitive nocturnal hypoxemia in OSA subjects (FIG. 1E). Consequently, overnight changes in SOD2 transcript levels in OSA vs. control subjects were significantly different (p=0.008) (FIG. 1F). The transcript level of catalase gene activity at night before sleep was similar in OSA and control groups (FIG. 1G). Here, catalase gene transcripts increased in response to overnight apneic sleep but did not change after a night of normal sleep (OSA vs. controls overnight % change: p=0.10) (FIG. 1H).

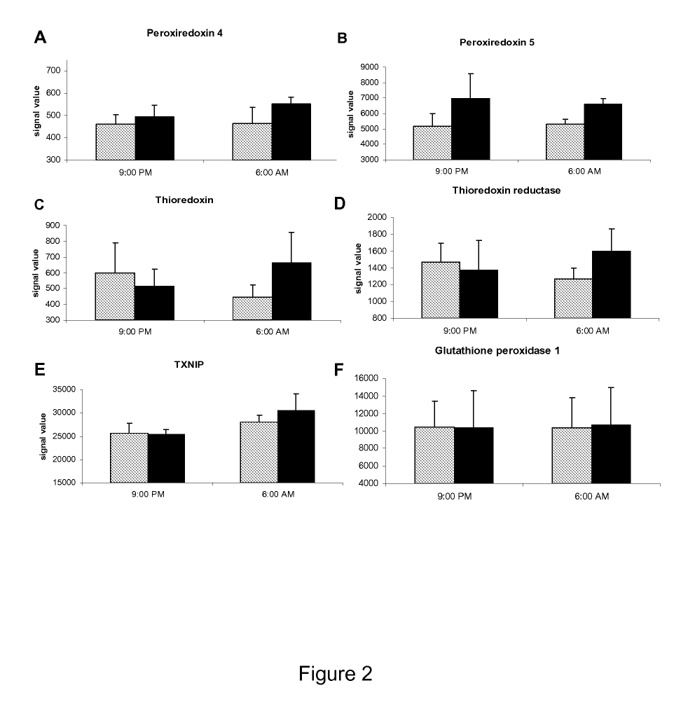

Peroxiredoxin 4 (FIG. 2A), peroxiredoxin 5 (FIG. 2B), thioredoxin (FIG. 2C), and thioredoxin reductase (FIG. 2D) gene transcript levels were not significantly different in OSA patients in comparison with controls at night. Even severe overnight hypoxemia in sleep apneics did not change these transcript levels significantly as compared to levels seen in control subjects after healthy normal sleep. No significant differences in thioredoxin interacting protein (TXNIP), an endogenous inhibitor of thioredoxin (FIG. 2E), nor in glutathione peroxidase gene transcript levels (FIG. 2F) were observed at any time point between the OSA and control groups.

### Genes Related to Cell Cycle and Proliferation

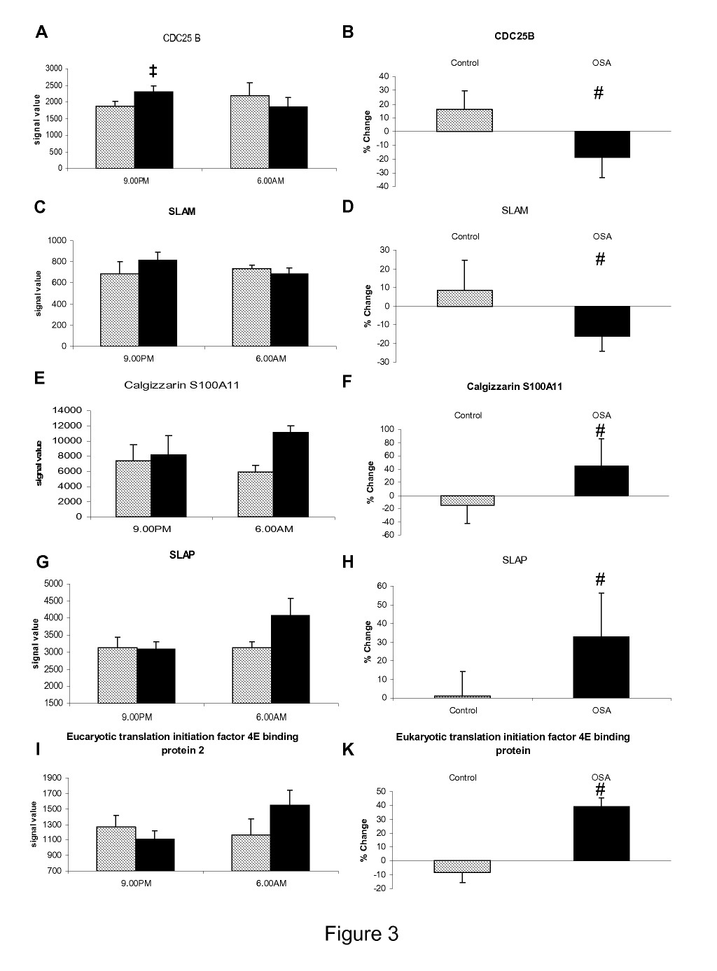

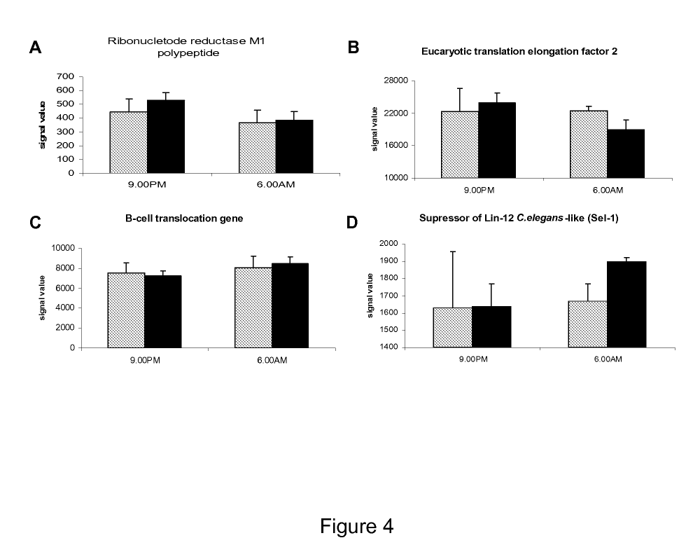

Microarray measures of gene transcript levels of several polypeptides involved in cell proliferation, activation, and growth were also determined. Both cell division cycle 25B (FIG. 3A) and signaling lymphocyte activating molecule (SLAM) (FIG. 3C) were higher at baseline (9 pm) in OSA than in controls. After overnight hypoxemia in OSA patients, however, gene transcripts levels of both of these decreased significantly (FIGS. 3B and 3D) in comparison to changes seen after overnight sleep in normal controls (Table 4). Significant changes after overnight sleep in OSA vs. control subjects were also observed in genes coding for calgizzarin (FIGS. 3E and 3F), Src-like adapter protein (SLAP) (FIGS. 3G and 3H), and eukaryotic translation initiation factor 4E binding protein 2 (FIGS. 3I and 3K). Changes observed in these gene transcript levels after overnight sleep in OSA patients exhibited significant differences as compared to control subjects. No significant differences were observed either at baseline measurements or overnight changes between control and OSA subjects in ribonucleotide reductase Ml polypeptide (FIG. 4A), eukaryotic translation elongation factor 2 (FIG. 4B), B-cell translocation gene (FIG. 4C), and supressor of Lin-12 C. elegans-like (Sell) (FIG. 4D).

The results provided herein demonstrate that nucleic acids encoding for enzymes involved in modulation of reactive oxygen species and their potential for cell damage can be differentially expressed in subjects with and without OSA and that the transcription of these nucleic acids can change acutely overnight during apneic sleep. These nucleic acids include those which are directly involved in lowering ROS levels, such as increased expression of catalase, and SOD2, along with increased basal expression of HMOX1. The results provided herein also demonstrate that nucleic acids that modulate the cell cycle can be altered in response to overnight apneic sleep, so as to potentially attenuate cell growth and proliferation. Expression of the nucleic acids identified herein can serve as an adaptive mechanism to limit cell death and damage in response to oxidative stress.

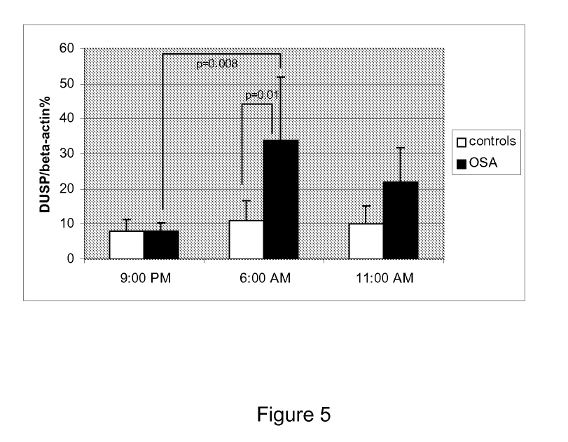

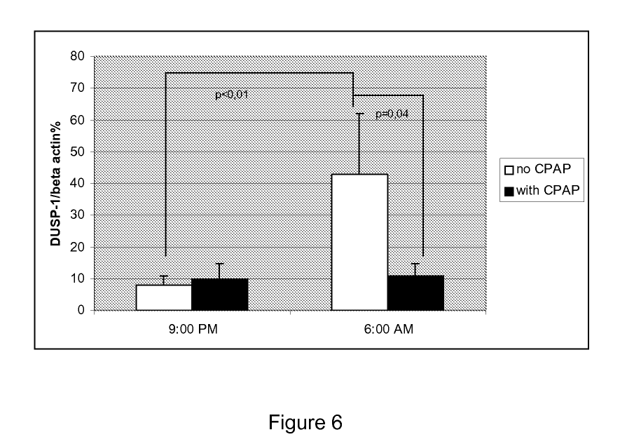

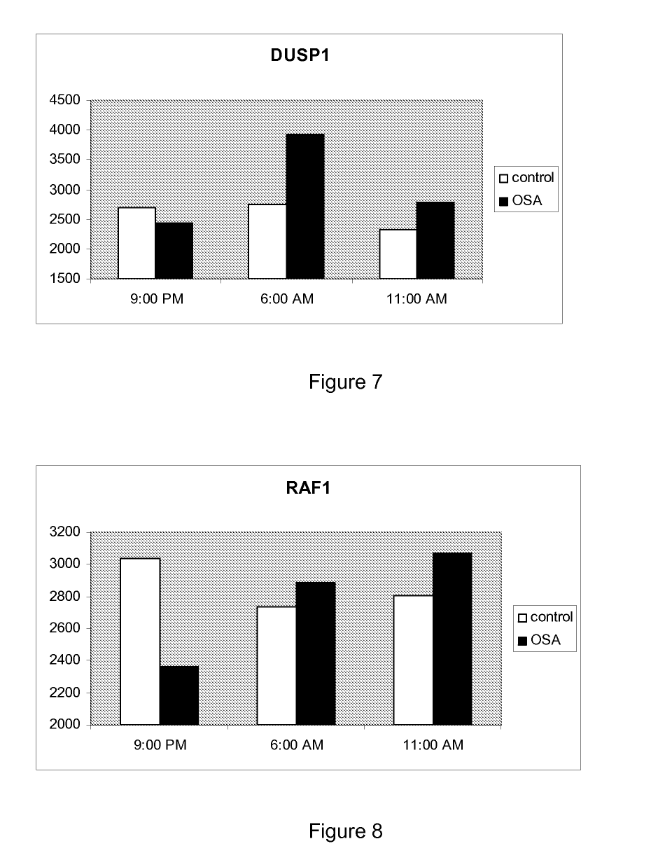

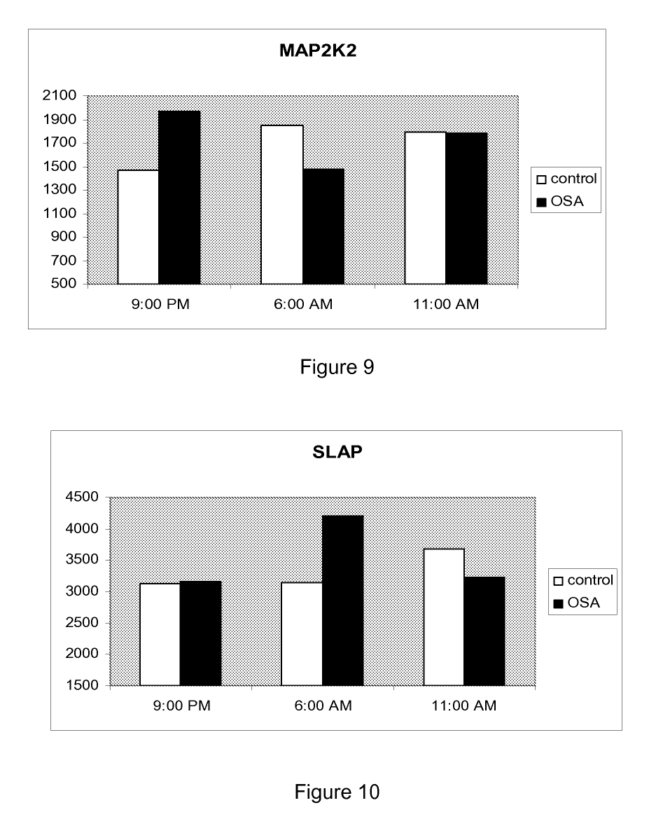

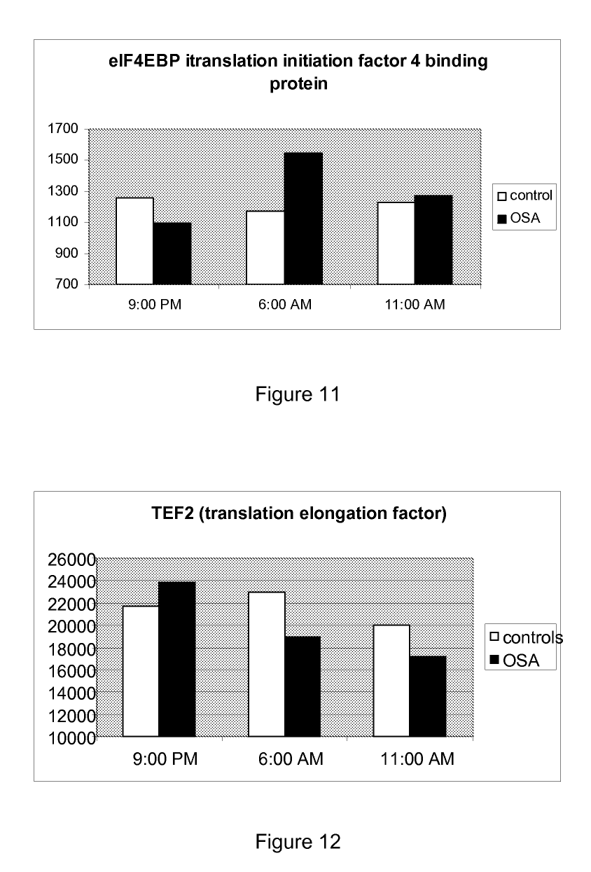

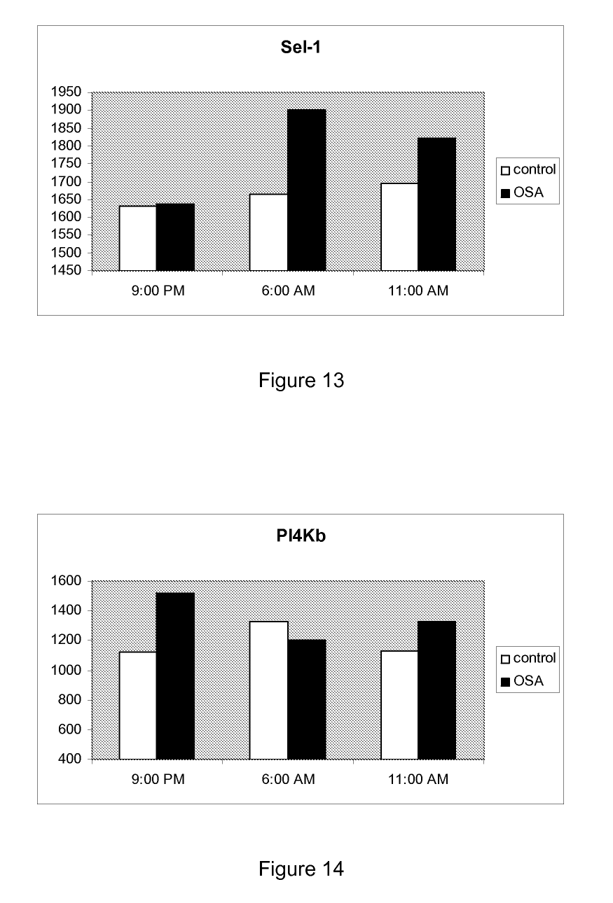

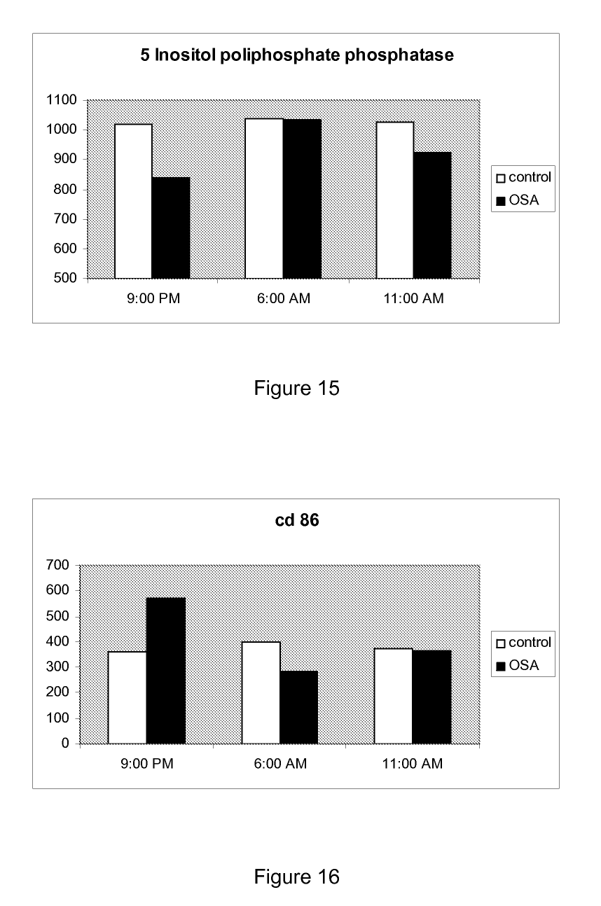

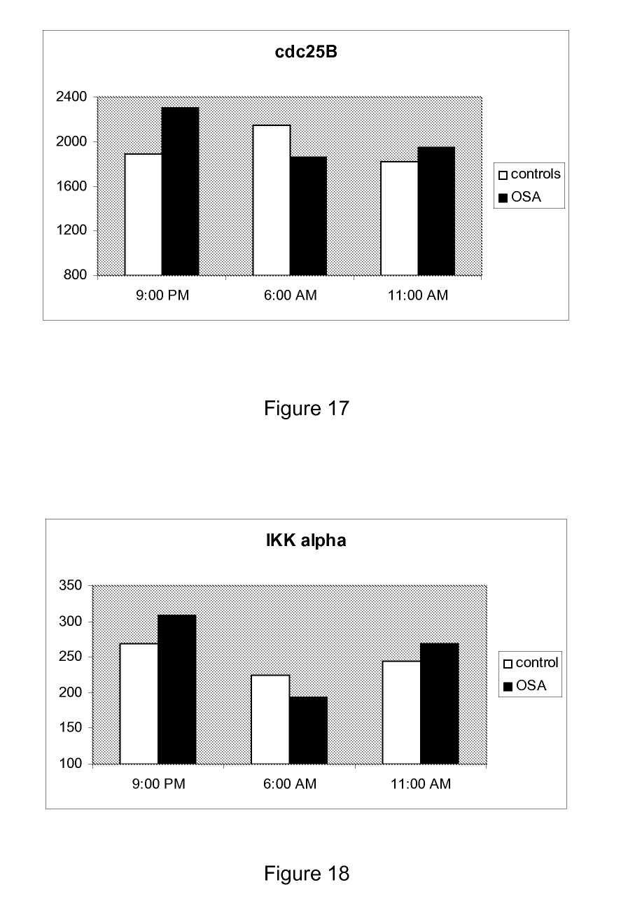

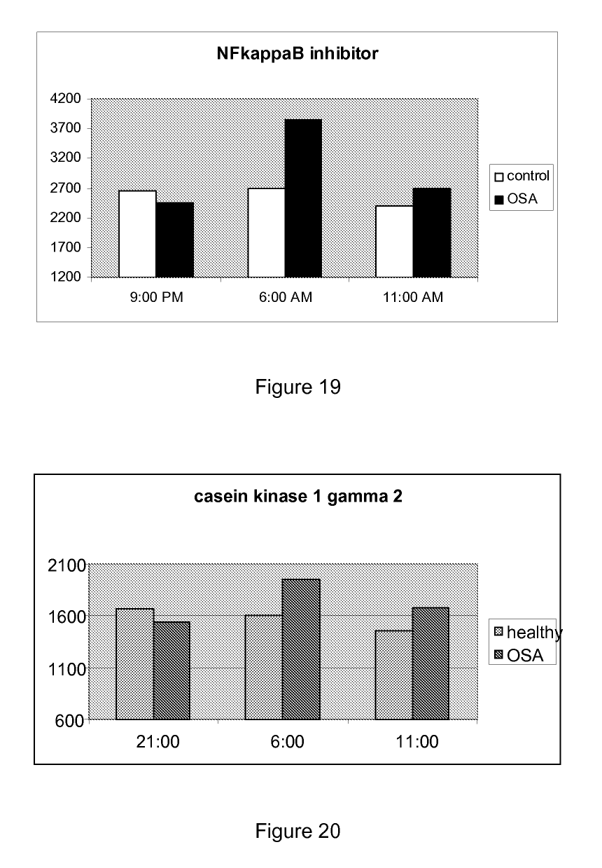

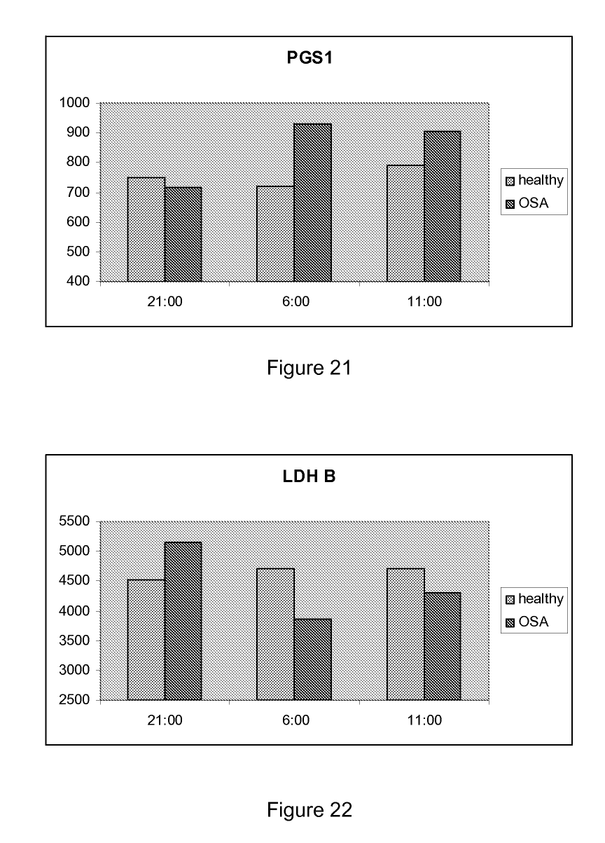

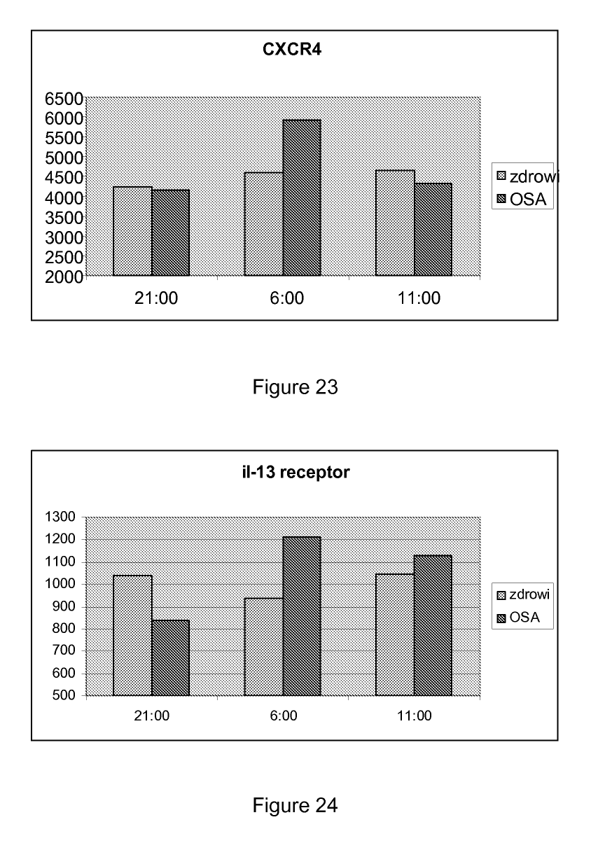

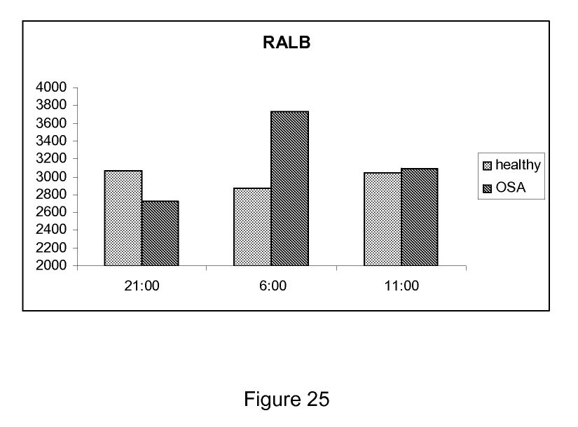

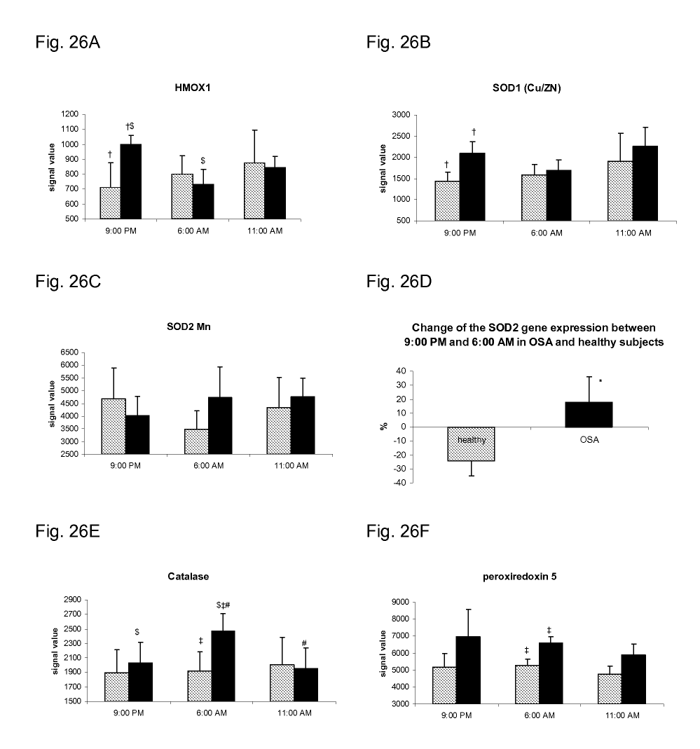

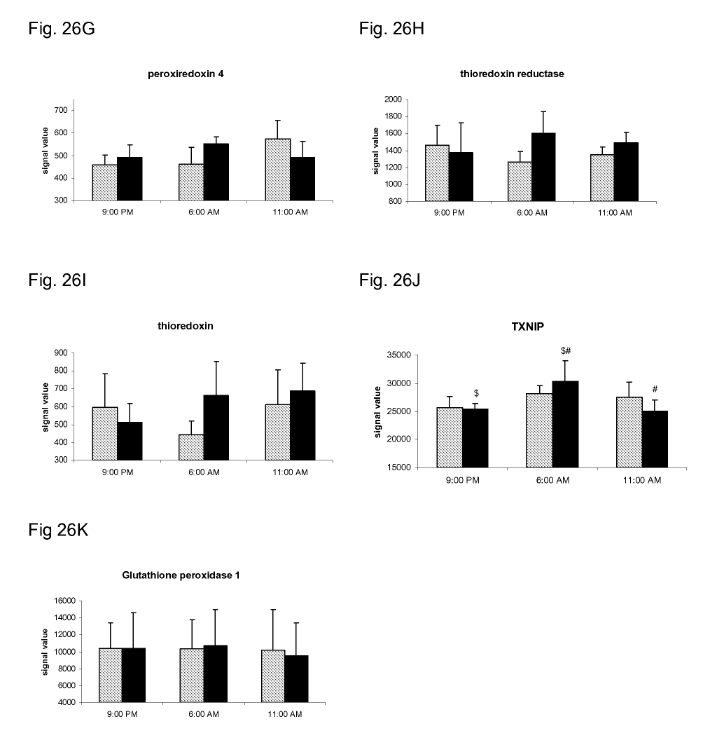

Measurements of dual-specificity phosphatase 1 (DUSP1) nucleic acid expression in blood obtained before sleep, after sleep, and after four hours of wakefulness from sleep were taken (FIG. 5). These measurements were obtained in normal subjects (people without sleep apnea), in patients with sleep apnea, and in patients with sleep apnea who received effective treatment with CPAP. These results demonstrate that in healthy normal subjects, measurements of DUSP do not change significantly after a night of normal sleep. Even when repeated after several hours of wakefulness from sleep, there is no change in DUSP. By contrast, in patients with untreated obstructive sleep apnea, measurements of DUSP increased significantly by the morning after overnight sleep. The increase in DUSP gradually returned towards baseline levels after several hours of wakefulness. By contrast, when these patients with sleep apnea are effectively treated with CPAP, measurements of DUSP did not change overnight (FIG. 6), and there was no significant decline during the course of daytime wakefulness. Thus, DUSP measurements demonstrate that biomarkers can be used to diagnose sleep apnea effectively and to monitor sleep apnea treatment effectiveness.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a mammal having sleep apnea, wherein said method comprises determining whether or not a mammal comprises a sleep apnea signature, wherein the presence of said sleep apnea signature indicates that said mammal has sleep apnea.

2. The method of claim 1, wherein said mammal is a human.

3. The method of claim 1, wherein said method comprises determining whether or not a blood sample from said mammal comprises said sleep apnea signature.

4. The method of claim 1, wherein said method comprises determining whether or not a urine, saliva, or perspiration sample from said mammal comprises said sleep apnea signature.

5. A method for identifying a mammal having sleep apnea, wherein said method comprises determining whether or not a mammal comprises a level of expression of a nucleic acid or a polypeptide indicative of sleep apnea, wherein the presence of said level indicates that said mammal has sleep apnea.

6. The method of claim 5, wherein said nucleic acid or said polypeptide is listed in Table 1.

7. The method of claim 5, wherein said mammal is a human.

8. The method of claim 5, wherein said method comprises determining whether or not a blood sample from said mammal comprises said level.

9. The method of claim 5, wherein said method comprises determining whether or not a urine, saliva, or perspiration sample from said mammal comprises said level of expression.

10. A method for assessing the effectiveness of a treatment for sleep apnea, wherein said method comprises determining whether or not the level of expression of a nucleic acid or polypeptide in a mammal being treated for sleep apnea changes during sleep, wherein a change in said level during sleep indicates that said treatment is ineffective.

11. The method of claim 10, wherein said nucleic acid or said polypeptide is listed in Table 1.

12. The method of claim 10, wherein said mammal is a human.

13. The method of claim 10, wherein said method comprises assessing a blood sample obtained from said mammal.

14. The method of claim 10, wherein said method comprises assessing a urine, saliva, or perspiration sample obtained from said mammal.
